(19) United States
(12) Plant Patent Application Publication
(10) Pub. No.: US 2002/0073454 P1
(43) Pub. Date: Jun. 13, 2002

(54) HYDRANGEA PANICULATA PLANT NAMED 'SHIKOKU FLASH'

(76) Inventor: Masato Yokoi, Kawaguchi City (JP)

Correspondence Address: Vincent G. Gioia, Christie, Parker & Hale, LLP, 5 Park Plaza, Suite 1440, Irvine, CA, 92614, US

(21) Appl. No.: 09/736,758

(22) Filed: Dec. 12, 2000

(51) Int. Cl. A01H 5/00

(52) U.S. Cl. PLT/250

(57) ABSTRACT

A new plant variety of Hydrangea paniculata named ‘Shikoku Flash’ having clear white mottled foliage growth throughout the growing season.

### BACKGROUND OF THE INVENTION

The present invention relates to a new and distinct variety of Hydrangea paniculata Sieb., which was discovered as a branch sport in a controlled planting of Hydrangea paniculata by the inventor, Dr. Masato Yokoi, in Ehime Prefecture, Shikoku, Japan. The varietal denomination of the new variety is ‘Shikoku Flash’.

The genus Hydrangea is included in the family Saxifragaceae which comprises about 80 genera and 1,200 species of herbs, shrubs, small trees, and woody climbers mostly temperate in origin. Hydrangea comprises approximately 23 species of deciduous or evergreen shrubs and vines originating in North America, South America and East Asia, many of which possess desirable ornamental characteristics. Recent taxonomical treatments sometimes isolate Hydrangea in a new family, Hydrangeaaceae.

Hydrangea paniculata is widespread and common throughout much of Japan and eastern and southern China. It generally has opposite, occasionally whorled, simple leaves, elliptic or ovate in shape with acuminate apex, 5-15 cm long and 5-10 cm wide. . Leaves are generally softly pubescent. Leaf color ranges from light to dark green, depending on light intensity and cultural conditions. The plant is deciduous. Flower color is white, often changing to pink as the season progresses Flowers of Hydrangea paniculata are borne in mostly conical or pyramidal panicles 15 to 20 cm long with a few white to pink sterile flowers each 1.75 to 3 cm wide. Fertile flowers. are numerous, blooming in summer and early fall.

### SUMMARY OF THE INVENTION

The new variety was discovered as a chance seedling in a controlled planting of Hydrangea paniculata. Asexual reproduction of the new variety by stem cuttings performed in Saitama Prefecture, Japan and Lewisberry, Pa. have confirmed that the distinctive characteristics of the new variety are stable and transmitted to succeeding generations.

### COMPARISON WITH PARENTS

‘Shikoku Flash’ is distinguished from its parent and all other varieties of Hydrangea paniculata known to the inventor by the pigmentation of the leaves, which exhibit a bright clear white variegation coloration throughout the growing season.

### BRIEF DESCRIPTION OF THE ILLUSTRATIONS

The accompanying photographic illustrations show a specimen of the new cultivar as true to color as is reasonably possible to make in an illustration of this character.

In the illustrations:

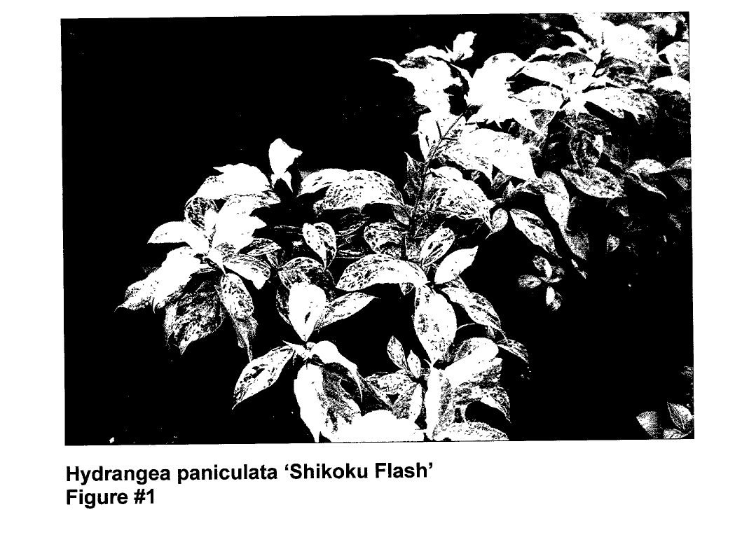

FIG. 1 illustrates the distinctive mottled, clear white variegated foliage coloration.

### DETAILED DESCRIPTION OF THE NEW VARIETY

‘Shikoku Flash’ has not been observed under all possible environmental, cultural, and light conditions. The following observations and descriptions are of plants grown in containers at Lewisberry, Pa. and Saitama Prefecture, Japan. Phenotypic expression may vary with light intensity and cultural and environmental conditions. In this description, color references are to The Royal Horticultural Society Colour Chart (1995), and terminology used in the color descriptions herein refers to plate numbers in this color chart.

Classification:

Botanical.—Hydrangea paniculata Sieb. ‘Shikoku Flash’

Parentage.—Branch sport of the variety Hydrangea paniculata.

Propagation.—By vegetative cuttings.

Plant:

Size.—Mature plant can grow to a height of 3 to 5 m.

Branching.—Freely flowering on new growth wood.

Habit.—Upright, mound forming deciduous shrub.

Leaf shape.—Ovate, acuminate apex.

Leaf margin.—Serrate.

Leaf texture.—Thick; pubescent to nearly glabrous on top; setose pubescent on bottom especially on veins.

Leaf color.—Color varies with intensity of sunlight and cultural conditions (see below). Color is generally yellow early in the growing season tending toward chartreuse as the foliage ages.

Upper side.—Clear white variegation near White Group 1 55D; medium green background near Green Group 141 B.

Petioles.—2.5 cm long.

Leaf size.—Up to approximately 15 cm long and 8 cm wide.

Texture and substance.—Flat, dull in appearance.

Hardiness.—USDA Zone 3 (−40° F. to −30° F.).

Infloresence:

Bloom Period.—Late summer until fall.

Flower Form.—Paniculate, upright conical.

Flower Arrangement.—Panicles; conical to pyramidal.

Flower Size.—About 1 to 20 cm long, 1.75 to 3 cm across.

Fertile flowers are numerous:

Color.—Clear white, near White Group 155D.

Shape of Inflorescence.—Conical.

Fragrance.—None noted.

Reproductive organs:

Stamens.—4 to 5.

Stigma.—1.

Fruit.—Extremely small, dust-like.

Sepalous Florets:

Number of sepals.—4 to 5.

Aspect of sepals.—1.

Size of sepals.—Approximately 75 mm wide.

I claim:

1. A new plant variety of Hydrangea paniculata of the variety substantially as shown and described.
